(19) United States
(12) Patent Application Publication
(10) Pub. No.: US 2009/0131782 A1
(43) Pub. Date: May 21, 2009

(54) Device for Heat Treating Moving Biological Tissues and Related Method

(76) Inventors: Chretien Moonen, Gradignan (FR); Charles Mougenot, Bordeaux (FR); Baudouin Dennis De Senneville, Bordeaux (FR)

Correspondence Address: Blakely Sokloff Taylor & Zafman, 1279 Oakmead Parkway, Sunnyvale, CA, 94085-4040, US

(21) Appl. No.: 11/992,902

(22) PCT Filed: Aug. 30, 2006
(86) PCT No.: PCT/EP2006/065832

§ 371 Date: Mar. 27, 2008

(30) Foreign Application Priority Data
Sep. 28, 2005 (FR) 0509885

(51) Int. Cl. A61B 5/055; A61N 7/02

(52) U.S. Cl. 600/411; 601/3

(57) ABSTRACT

This invention concerns a device for heat treating a moving target area of biological tissues, comprising calculation means (6) for estimating the position of a target area using a measurement signal of the target area, characterized in that it comprises control means (7) for positioning a treatment focal point (P) in the target area based on the estimated position and a positioning time lag between a measurement of the measurement signal of the target area and the positioning of the treatment focal point (P), so as to compensate the movement of the target area during the positioning time lag.

The invention also concerns a related heat treating method.

### FIELD OF THE INVENTION

The invention relates to the field of hyperthermic treatment of biological tissues.

### STATE OF THE TECHNOLOGY

The hyperthermic therapies are techniques that are presently used for the local treatment of biological tissues. It consists of heating a target area of biological tissue using a source of energy (laser, microwaves, radio waves, ultrasound).

In general, therapies using local hyperthermia treatment enable medical procedures with a minimum invasiveness. Among the different types of energy that are used, focused ultrasounds (FUS) are particularly interesting since they heat a target area noninvasively and deep into the tissue.

During the treatment, the temperature of the target area and its surrounding environment must receive precise and continuous control. For the treatment device to be completely non-invasive, it is possible, for example, to use Magnetic Resonance Imaging (MRI) to obtain a precise map showing the distribution of the temperature as well as detailed anatomic data.

The non-invasive systems are nevertheless fixed position system while biological tissues, and therefore the target areas to be treated, present all different types of movement.

Document U.S. Pat. No. 5,938,600 presents a treatment method for biological tissue in motion and a related method. According to this method it is possible to automatically determine the movement in a target area in motion using an MRI system and then to generate a signal representing the motion thus determined. This signal allows an ultrasound device to radiate out into a focal area determined by the target area in motion. However, this type of treatment method only allows for a partial correction of the movement of the target area, and some zones of biological tissue may receive unwanted radiation.

One of the objectives of the present invention is therefore to provide an improved device for treating biological tissues in motion, with the device making it possible to solve at least one of the limitations mentioned above.

### DESCRIPTION OF THE INVENTION

To this end is provided a heat treatment device for treating a target area in motion of a biological tissue, this device comprising calculation means for estimating the position of the target area using a measurement signal from the target area, characterized in that it further comprises control means for positioning a treatment focal point in the target area based on the estimated position and a positioning time lag between a measurement of the measurement signal and the positioning of the treatment focal point, in order to compensate the movement of the target area during the positioning time lag.

Some preferred but non-limitative aspects of the heat treatment device are the following:
the positioning time lag comprises a latency time due to the estimation of the position of the target area using the calculation means, so that the control means are adapted for compensating the movement of the target area during the latency time
the positioning time lag comprises a prediction time of the movement of the target area, so that the control means are adapted for predicting and compensating the movement of the target area during the prediction time.
the treatment device further comprises measurement means of the latency time to transmit to the control means.
the treatment device further comprises modeling means for modeling the movement of the target area based on a series of measurement signals of the target area; the modeling of the movement may be periodic.
the modeling means comprise means to provide a spatial position of the target area based on a temporal position, with the spatial and temporal positions defining the position of the target area.
the calculation means comprise means to determine an estimated spatial position of the target area using a algorithm for processing of the measurement signal of the target area.
the calculation means comprise means to determine, according to the modeling of the movement of the target area, an estimated temporal position of the target area corresponding to the estimated spatial position.
the control means comprise means to position the treatment focal point in accordance with a real temporal position of the target area, the real spatial position being based on the real temporal position according to the modeling of the movement of the target area, the real temporal position corresponding to the estimated temporal position enhanced by the latency time.
the control means further comprise means to position the treatment focal point between successive estimations performed by the calculation means of a first and second temporal position estimated respectively from a first and second measurement signal of the target area, in order to predict the movement of the target area.
the control means comprise means to position the treatment focal point according to a predicted spatial position, the predicted spatial position being a factor of a predicted temporal position according to the modeling of the movement of the target area, the predicted temporal position corresponding to the estimated temporal position enhanced by the latency time and the prediction time.
the calculation means comprise means to determine an estimated displacement vector field of the target area using an algorithm for processing of the measurement signal of the target area.
the control means comprise means to position the treatment focal point in the target area as a factor of the estimated displacement vector field.
the measurement signal of the target area is measured by imaging means to provide an anatomic image of the target area.
the imaging means may further comprise means to provide a phase image of the target area using the measurement signal of the target area, for monitoring temperature variations of the target area using a reference phase image.
the calculation means may further comprise means to modify the reference phase image to correct a disturbance of the temperature due to a movement of the target area.
the calculation means may further comprise means to modify the reference phase image using for example stored phase images.
the treatment device further comprises regulation means of a radiation applied on the treatment focal point in the target area so that the spatial distribution of temperature of the target area should conform with a setting for the spatial distribution of temperature; the regulation means comprise means to regulate the applied radiation as a factor of the spatial distribution of temperature in the target area and a setting for the spatial distribution of temperature, in accordance with a regulation equation comprising a Proportional-Integral-Derived term.
the heat treatment of the target area is non-invasive.

I is further provided a heat treatment method of a moving target area of biological tissue, characterized in that it comprises the steps of:
Measuring the target area for obtaining a measurement signal of the target area,
Estimating a position of the target area based on the measurement signal of the target area using calculation means,
Positioning a treatment focal point in the target area using control means, in based on the estimated position and the positioning time lag between the measurement of the measurement signal of the target area and the positioning of the treatment focal point, so as to compensate the movement of the target area during the positioning time lag.

Some preferred but not limiting aspects of the heat treatment device are the following:
the positioning time lag comprises a latency time due to the estimation of the position of the target area using the calculation means (6), so as to compensate the movement of the target area during the latency time.
the positioning time lag comprises a prediction time of the movement of the target area, so as to predict and compensate the movement of the target area during the prediction time.
the method further comprises the step of measuring the latency time in real time using measurement means (10).
the method further comprises the step of modeling the movement of the target area based on a series of measurement signals of the target area, during which a period for modeling the movement of the target area may be determined.
the position of the target area is modeled using a spatial position as a factor of a temporal position.
the estimation of the position of the target area comprises the steps of:
   Determining the estimated spatial position of the target area using an algorithm for processing the measurement signal of the target area, and
   Determining, according to the modeling of the movement of the target area, an estimated temporal position corresponding to the estimated spatial position.
the positioning of the treatment focal point comprises the steps of:
   Determining a real temporal position of the target area, the real temporal position corresponding to the estimated temporal position enhanced by the latency time,
   Determining a real temporal position of the target area, according to the modeling of the movement of the target area, based on the real temporal position, and
   Positioning the treatment focal point according to the real spatial position.
the positioning of the treatment focal point comprises the additional steps of:
   Determining a predicted temporal position of the target area, the predicted temporal position corresponding to the estimated temporal position enhanced by the latency time and a prediction time,
   Determining a predicted spatial position of the target area, according to the modeling of the movement of the target area, based on the predicted temporal position, and
   Positioning the treatment focal point based on the predicted spatial position.
the additional steps for positioning the treatment focal point (P) are repeated until a new estimated temporal position is determined.
the method further comprises the step of determining an estimated displacement vector field of the target area using an algorithm for processing the measurement signal of the target area; the treatment focal point is positioned in the target area based on the estimated displacement vector field.
the measurement signal of the target area provides an anatomic image of the target area for positioning the treatment focal point, and may further provide a phase image of the target area.
the method further comprises the steps of:
   Determining a reference phase image;
   Comparing the acquired phase image and the reference phase image for monitoring the temperature variations in the target area.
a reference phase image is determined from the stored phase images.
the method further comprises the step of regulating the radiation applied to the treatment focal point (P) in the target area so that the spatial distribution of temperature in the target area conforms with a setting for the spatial distribution of temperature.
the radiation applied is regulated based on the spatial distribution of the temperature in the target area and the setting for the spatial distribution of temperature, according to a regulation equation comprising a Proportional-Integral-Derived term.
the treatment method is performed non-invasively.

### DESCRIPTION OF THE FIGURES

Other characteristics and advantages become apparent in the description here below, which is purely illustrative and not limitative and which must be read with due attention to the figures included in the annex among which are:

FIG. 1 is a schematic representation of the treatment device according to the invention;

FIGS. 2a to 2e illustrate a method of resetting the images;

FIG. 3 is a schematic representation of the assessment platform of the treatment device according to the invention;

FIGS. 4a and 4b are representations of the movements of the target area according to a first assessment of the correction of the position of the treatment focal point according to the invention;

FIGS. 5a and 5b are representations of the movements of the target area according to a second assessment of the correction of the position of the treatment focal point according to the invention;

FIGS. 6a and 6b are representations of the movements of the target area according to a third assessment of the correction of the position of the treatment focal point according to the invention;

FIGS. 7a and 7b are representations of the movements of the target area according to a fourth assessment of the correction of the position of the treatment focal point according to the invention;

FIGS. 8a to 8f illustrate a first assessment of the correction of the temperature measurement and the position of the treatment focal point by the treatment device according to the invention;

FIG. 9 is a representation of the spatial distribution of temperature according to a first assessment illustrated in FIGS. 8a to 8f;

FIG. 10 is a representation of the temporal changes in temperature according to a first assessment illustrated in FIGS. 8a to 8f;

FIGS. 11a to 11f illustrates a second assessment of the correction of the temperature measurements and the position of the treatment focal point by the treatment device according to the invention;

FIG. 12 is a representation of the spatial distribution of temperature according to a second assessment illustrated in FIGS. 11a to 11f;

FIG. 13 is a representation of the temporal change in temperature according to a second assessment illustrated in FIGS. 11a to 11f;

FIGS. 14a and 14b represent the images necessary for resetting the images during periodic elastic movement;

FIGS. 15a to 15f illustrate the third assessment of the correction of the temperature measurement and the position of the treatment focal point by the treatment device according to the invention;

FIG. 16 is a representation of the spatial distribution of temperature according to a second assessment illustrated in FIGS. 15a to 15f;

FIG. 17 is a representation of the temporal changes in temperature according to the second assessment illustrated in 15a to 15f;

FIGS. 18a and 18b are graphs illustrating a first assessment of temperature control by the treatment device according to the invention correcting the position of the treatment focal point;

FIGS. 19a to 19d are MRI images illustrating the first assessment of temperature control;

FIGS. 20a to 20d are representations of the spatial distribution of temperature according to the first assessment of temperature control;

FIGS. 21a and 21b are graphs illustrating a second assessment of temperature control by the treatment device according to the invention correcting the position of the treatment focal point;

FIGS. 22a to 22d are MRI images illustrating the second assessment of temperature control;

FIGS. 23a to 23d are representations of the spatial distribution of temperature according to the second assessment of temperature control.

### DETAILED DESCRIPTION OF THE INVENTION

### Description of the Device for Treating Biological Tissues in Motion According to the Invention and its Functioning

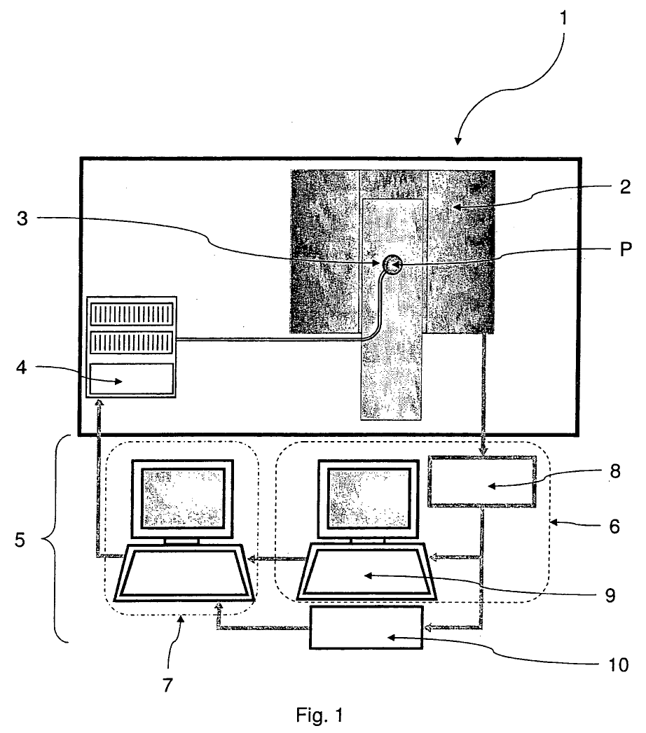

FIG. 1 represents a treatment device for biological tissues comprising measurement means for a target area of biological tissue to be treated so as to provide a measurement signal of the target area to be used in characterizing the target area (its movement for example). For non-invasive treatment, one may for example use as measurement means, MRI 2 imaging means to provide images of the target area of the biological tissue to be treated. This MRI 2 imaging apparatus may for example comprise a 1.5 Tesla magnet and shall be able to provide modular images (or anatomic images) and phase images (or heat images), that are two- or three-dimensional, of the target area of tissue to be treated. By preference, an MRI imaging apparatus shall be selected with a millimeter spatial resolution, 1° C. precision and a second temporal resolution.

The treatment device 1 according to the invention further comprises the energy generating means in the form of a matrix transducer 3 and a multichannel generator 4 feeding the matrix transducer 3. The transducer 3 is integrated into the magnet bed of the MRI imaging apparatus 2 and it serves to focus an ultrasound wave in the direction of a treatment focal point P of the target area. We shall select, for example, a 256 element matrix transducer serving to focus an ultrasound wave of 1.5 MHz on a point with the dimension of a wavelength, which is about 1 mm. The acoustic pressure at the treatment focal point P and its position have adjustable amplitude and delay time of the signal emitted by the multi-channel general 4. As such, the position of the treatment focal point P may be adjusted for a volume of 15×15×30 mm³ every 100 milliseconds.

The matrix transducer 3 may be replaced by a simple transducer. In such a case, the displacement of the treatment focal point must be done mechanically, with a hydraulic displacement system for example, and shall therefore present a slower response time than for a matrix transducer.

The heat treatment device 1 further comprises a managing unit 5 which is able to receive at its input, data originating from the MRI imaging apparatus 2 and, as a factor of these data, to control the multichannel generator 4 to modify the position of the treatment focal point P via the matrix transducer 3.

Indeed, the measurements acquired in the interior of the magnet of the MRI imaging apparatus 2 are transmitted to calculation means 6 of the managing unit 5. These calculation means 6 are for treating the data originating from the MRI imaging apparatus 2 and transmitting them to control means 7 of the managing unit. The control means 7 use data originating from the calculation means 6 and transmits the coordinates and the power of the next target points to the multichannel generator 4, by the intermediary of fiber optics for example. The multichannel generator 4 produces and amplifies out of phase ultrasound electric signals so that the matrix transducer 3 emits a focused ultrasound wave at the select point. The increase in temperature induced at the interior of the treatment focal point P makes it possible to obtain the desired heat dose, necessary to obtain a necrosis for example.

The calculation means 6 are therefore used for processing the data emitted by the MRI imaging apparatus 2, in particular in order to assess the position of the target area.

The first calculation step consists in converting the measurements acquired in the interior of the magnet of the MRI 2 imaging apparatus. For this, an image reconstructor 8 is used for example to perform a Fourier transformation and filtering of different data originating from the MRI imaging means 2 so as to reconstruct an image of the target area.

These images allowing for the visualization of the target area being studied are then transferred using the processing means 9 for assessing the position of the target area. Indeed, the biological tissue being considered are in motion, and the target area to be treated is also in motion, and it is therefore necessary to have a precise estimate of the displacement of the target area in order to be able to correct the position of the treatment focal point P of the target area over time, as a factor of the motion of the target area. This type of correction permits more precise radiation of the tissue, and as a consequence, more efficient treatment.

There are various imaging techniques for assessing the motion of biological tissues using MRI images; it is possible for example to estimate the displacement of these tissues using anatomic images reconstructed from MRI imaging 2 data. Several image resetting algorithms match the coordinates of each image point being reset with the coordinates of their corresponding images on a reference image.

As part of the constraints due to real time processing, the practical procurement of three-dimensional images is quite difficult due to the technical limits of the sequence acquisition that is necessary. An alternative approach consists in estimating the displacement on two dimensional images generated by objects in motion in a three dimensional space. The position and orientation of the cut planes should be selected so as to have the axis of motion as part of the image. If the displacements estimates of the target area described herein are based on two-dimensional images, the invention is not limited to the estimates of the two dimensional displacements fields, and may therefore be easily applied to the estimates of the three dimensional displacement field.

Among the different algorithm for resetting images, several approaches stand out. Some algorithms are indeed based on the estimate of a global transformation in the image while others use information relative to local transformation of the image. The resetting method that is most efficient isn't the one which provides the most similarity between the reference image and the reset image but rather the one which provides an estimate of the displacement field that is closest to the real motion of organs. The process that is used therefore consists in first using the dominant total movement, present in the image, then to refine locally the displacements of the organs. The goal is to assess the motion using a local approach based on the results obtained by a global approach.

The search for optimum parameters of a global affine transformation may for example be performed using an optimization of the least-squares sense. A hierarchical approach of the algorithm of Horn & Schunck then allows for making a good estimate of the local displacement of the tissues since the regularity constraint, imposing the displacement vectors to be similar for adjacent pixels, coincides with the real motion of the tissues.

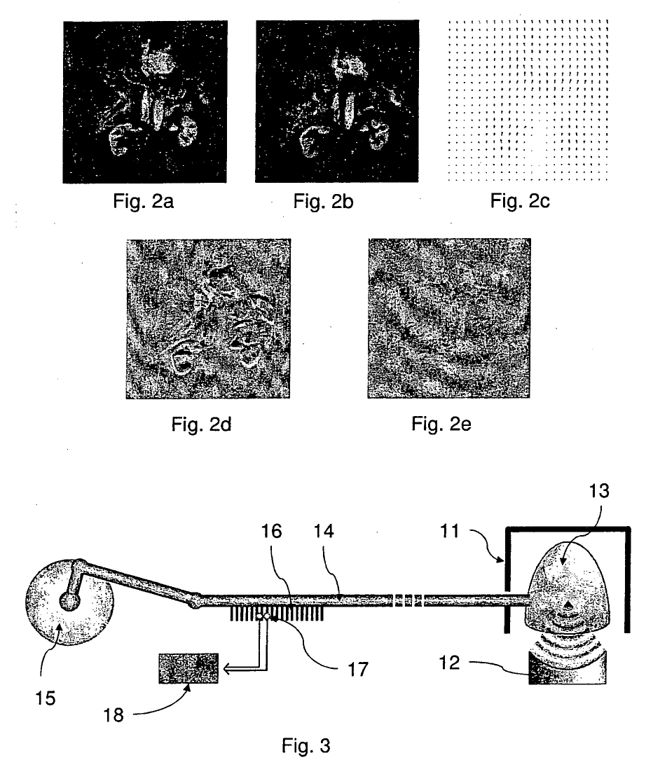

FIGS. 2a and 2e illustrate this image resetting process. FIGS. 2a and 2b are anatomic images of a free-breathing human abdomen, respectively obtained at the start and at the end of expiration. FIG. 2c represents the two dimensional vector field assessed using an image resetting algorithm. FIG. 2d is obtained by taking away a reference image (the image in FIG. 2a, at the start of expiration) from the resetting image (image in FIG. 2b, at the end of expiration). FIG. 2e represents an image obtained by taking away a reference image from the resetting image, which shows that the resetting of the image was correctly performed.

These different processing techniques shall therefore make it possible, using images resulting from data transmitted by MRI imaging 2 means, to determine numerous data concerning the movements of the target area to be processed, as well as an assessment of the position of the target area.

When biological tissue is handled in a medical situation, it may undergo various displacements and the position of the treatment focal point P must be readjusted so as to be always located at the interior of the target area initially defined. The managing unit 5 of the treatment device 1 according to the invention is therefore used to correct the different movements that may happen to the biological tissue undergoing treatment. The motion of biological tissue and therefore of the target area, may principally be classed into two categories, depending on their temporal occurrence. Indeed, there are incidental movements as for example occurs when a muscle contracts, and there are also so-called periodic movements, which are linked for example to the breathing cycle or the heart cycle, as for example is the case with the liver and the kidneys. We may notice that each of these movements may be seen as rigid movements (comprising both a translation and/or rotation movement) even though they are in fact more complex elastic movements.

For each of these two types of movements (incidental or periodic) it is necessary to adapt the corrective strategy being used.

Indeed, in order to correct incidental movements for example, it is proper to use for example the estimated displacement vector field using the calculation means as of the last anatomic image available. Thanks to this estimate of the displacement vector field, the position of the treatment focal point P is corrected by following the estimates displacement in relation to its location.

The characteristic repetition of a periodic movement allows for adapting a corrective strategy used for incidental movements so that the positioning of treatment focal point P may be more precise. Indeed, the preceding strategy demands that the real movement of the target area is equivalent to the estimated movement on the last acquired image. According to this hypothesis, the time interval is considered negligible between the transmission of the data originating from the MRI imaging means 2 and the availability of information present in this image. This latency time between the transmission of data originating in the MRI imaging means 2 and the correction of the positioning of the treatment focal point P is typically composed of the acquisition time, the image reconstruction time, the transfer time, and the calculation time for assessing the position of the target area. This time lag being typically 2 seconds, and the period of a respiratory movement being 5 seconds, the strategy adopted to correct the incidental movements generates an estimated movement which is nearly in a phase in opposition to the real movement of the target area. As a consequence, this correction strategy may double the positioning error of the treatment focal point P if it is used to correct periodic movements.

For these reasons, it is indispensable to correct the periodic movement by quantifying the latency time that requires compensation. A corrective strategy may for example be based on the analysis of the global dominant movement variation present in the image.

Compensation for a periodic movement requires several steps which are described here below.

It is important to first make a model analysis of the dominant movement of the target area to be treated. This modeling step is performed using modeling means which are activated during the pretreatment phase during which the sequence of biological tissue movements of the target area are analyzed. Due to the respiratory cycle in particular, the target area follows a movement marked by a certain periodicity. Since the respiratory cycle is not entirely regular, the average number of periods acquired during the pretreatment step is measured in order to establish an average period. Each of these periods is adjusted to the established average period in order to obtain a precise sampling of the respiratory cycle.

We may then translate the dominant movements of the target area into periodic movements which breakdown into a Fourier series of order N as per the following equation:

$$M ( t ) = {!}_{n = 0}^{N} {a}_{n} cos ( n \# t ) + {b}_{n} sin ( n \# t ) \qquad [ Equation 1 ]$$

In this equation, aₙ and bₙ are the components of each harmonic. In practice, N=3 is a good value for efficient modeling for an average period. The harmonics that are higher than this have indeed an amplitude which is below 0.1 millimeter.

The determination of the coefficients aₙ and bₙ is done based on the method of the least-squares with the different points K acquired during the pretreatment step:

$${a}_{n} = \frac{{!}_{i = 1}^{K} M ( {t}_{i} ) cos ( n {t}_{i} )}{{!}_{i = 1}^{K} {cos ( n {t}_{i} )}^{2}} and {b}_{n} = \frac{{!}_{i = 1}^{K} M ( {t}_{i} ) sin ( n {t}_{i} )}{{!}_{i = 1}^{K} {sin ( n {t}_{i} )}^{2}} \qquad [ Equation 2 ]$$

According to one of embodiment of this invention, the modeling means are further able to model the movement of the target area during the treatment step.

Furthermore, we may use modeling means which can model non periodic movements, these movements being characterized in that they extend over a relatively long period of time, in comparison to incidental movements.

The movement of the target area being modeled, we may be much more precise in our correction of the position of the treatment focal point P.

The first step for repositioning of the treatment focal point P consists in analyzing the measurement signal of the MRI imaging means 2, in the aforementioned manner for example, in order to obtain information on the position of the target area.

It is important to first determine the spatial position of the target area based on the anatomic image originating from the MRI imaging means 2. The spatial position originates from the analysis of displacement D of the target area between an image and a reference image.

The second step consists in determining the temporal position corresponding to the image of the target area. This temporal position tᵢ of the last image is determined as a factor of the spatial position previously determined and as a consequence, by determining the solution of the equation D=M(tᵢ), a factor of the displacement of the dominant D corresponding to the average period.

However, the trigonometric polynomial M(t) modeling of the movement of the target area has several roots; a lack of proper determination is therefore a factor concerning the ascendant or descendent period of the cycle. In order to reverse this lack of determination, it is therefore necessary to refer to the stored displacement data for the target area. One may then calculate the Euclidian distance between the dominant displacement of the L last dynamics (for example, setting L at 5) and the trigonometric polynomial in order to temporally locate the position of the target area on the acquired image in a precise and stable manner.

Once the temporal position is determined, the position of the target area in relation to the mobilization of the movement is precisely defined. Nevertheless, this assessed position corresponds to the position of the target area at the moment when the MRI imaging means 2 have acquired an image, and not at the real position of the target area. Indeed, in the latency time which was taken to transfer and process the data originating from the MRI imaging means 2, the movement of the target area does not cease. The real position of the target area at the moment when the position information is available is different from the assessed position. It is therefore necessary to compensate the movement which took place during this latency time.

Therefore, the control means 7 of the managing unit 5, for positioning the treatment focal point P, are able to determine the real position of the target area and to position the treatment focal point P as a factor of this real position.

At first, the control means determine the real temporal position which corresponds to the assessed temporal position to which is added the latency time necessary to calculate the position.

Once the real temporal position is determined, the modeling of the movement of the target area is used to determine the corresponding real spatial position and position the treatment focal point P as a factor of this real spatial position.

To determine the real temporal position, we may for example consider that the latency time is constant by making it equal to the average value of the latency time s measured during the pretreatment step.

According to another embodiment of this invention, the treatment device 1 shall be provided with measurement means 10 to measure precisely the value of the latency time of each dynamic necessary to assess the position of the target area using the data of the MRI imaging means 2. The treatment device 1 may for example comprise a microcontroller 10 to make a time measurement of the time between the moment when the data of the MRI imaging means 2 are received by the calculation means 6 and the moment when the assessed temporal position is determined. As we shall see later on, the use of such measurement means 10 allows for a precise compensation of the latency time, given that the latter is very dependent on the different calculation times needed for the calculation means, these calculation times possibly varying from one assessment to another, given that the work capacity may vary from one dynamic to another.

Other than the compensation of the movement of the target area during the latency time, the control means 7 are able to predict the motion of the target area.

There is in fact a certain time lag between the two successive dynamics of data acquisition by the MRI imaging means 2, and therefore there is a time lag between two successive determinations of the real spatial position of the target area. Nevertheless, the knowledge of the assessed temporal position of the target area and of the latency time, measured for example by a microcontroller 10, makes it possible to predict the dominant displacements expected up until the processing of the next MRI data thanks to the polynomial modeling of the dominant movement of the target area.

If one tries to reposition the treatment focal point P in the target area despite the fact that a certain time has lapsed since the last determination of the real spatial position of the target area, time which is called the << prediction time >>, it is sufficient to determine the predicted spatial position corresponding to the modeling of the movement of the target area at a predicted temporal position, and this predicted temporal position is equal to the last assessed temporal position enhanced by the latency time and the prediction time.

We shall preferably select several prediction times in order to reposition the treatment focal point as often as possible. Preferably, the control means shall reposition the treatment focal point P with a prediction until a new assessed temporal position shall be determined for the calculation means 6.

It must be noted that in all cases where the latency time is weak, due to the performance of the calculation means 6 for example, one may decided to ignore it. In this case, the control means 7 shall compensate the movement of the target area by positioning the treatment focal point P taking into account the prediction lag alone (the latency time is considered as zero).

For the tissue movements considered as rigid, the knowledge of the global dominant movement is sufficient to determine the displacement field for the overall object and the modeling of the dominant movement is therefore sufficient as well.

On the other hand, for more complex tissue movements, such as elastic movements, a movement atlas shall be built. This atlas contains the stored memory of the displacement fields (such as the one present in FIG. 2c) assessed during a pretreatment step performed before the medical procedure. During the procedure, the atlas searches for the global dominant movement that is closest in order to deduce thereof the local associated displacement fields. It's on the basis of the displacement fields that the position assessments shall be made.

Furthermore, in order to increase the stability of the system, it is important to reduce the latency time by a maximum. In order to do this, we may use other measurement means for delivering real time measurement signals of the target area (such as for example navigator echoes or ultrasound echoes) in order to define the dominant movement of the target. This dominant movement may be used to directly correct the rigid movement or to permit the selection if displacement fields from the atlas created from the RMI images.

The real time measurement signals of the target area obtain an assessment of the movement with excellent temporal resolution and the RMI measurement signals provide a precise spatial assessment of the movement, an approach which combines the different measurement signals of the target area and which would thus make use of the spatial and temporal advantages of each movement measurement tool.

According to another embodiment of the invention, the MRI imaging means 2 of the treatment device are not only able to provide data of the anatomic image the target area, but they are also designed to provide data of the heat image of the target area. Such a heat image makes it possible to represent the spatial distribution of temperature of the target area.

Magnetic resonance imaging is indeed based on the detection of the magnetic properties of the protons contained in the water molecules of the body. The system for constructing images by magnetic resonance associates each volume unit (voxel) of the area being studied with a complex number Me^{iφ}, where M is the molecule and φ is the vector phase of macroscopic magnetism. The module M provides information about the anatomy and makes it possible to construct the so-called anatomic image. The principle of MRI-guided temperature measurement is based on performing a dynamic image acquisition by analyzing the contrast variations for calculating the temperature mapping. The method that is most commonly used to perform a dynamic temperature measurement, especially at the higher order of value (1.5 Tesla), is to compare the contrast of the phase images obtained at different times. Under certain well-defined conditions (without movement artifacts or susceptibility thereof) a difference in phase between two consecutive images is directly proportional to the difference in temperature:

!=#..B₀.!T.T_{E} [Equation 3]

where ΔT is the difference in temperature, γ is the gyromagnetic ratio (#42,58.2! MHz/T), α the temperature coefficient (=0.01 ppm/K), T_{E} the echo delay, B₀ the magnetic induction of the magnet. This calculation is performed for each image pixel in order to obtain a temperature map.

With this monitoring of the temperature measurement of the target area, it is then possible to determine the heat dosage, in order to assess the degree of necrosis of the biological tissue to be treated.

Nevertheless, this monitoring method of temperature measurements is sensitive to possible movements of the target area of the biological tissue. It is therefore necessary, for a precise monitoring of temperature variations, to correct the temperature measurement artifacts that are generated by the different movements of the target area, whether these are incidental or periodic.

Indeed, if a movement occurs incidentally between the times tₙ₋₁ and tₙ, the calculated temperature maps following this incidental movement will be false. A simply method consists in taking the phase image acquired at time tₙ (φₙ) as the new reference phase image. The temperature map at the time tᵢ with i>n, is calculated with the following equation:

#Tᵢ=#T′+(!ᵢ!ₙ)k [Equation 4]

where ΔT′ is the n−1 map of the temperature after correcting for movement.

In order to correct the temperature artifacts generated by the periodic movements, it is possible to analyze the disturbance of the temperature images as a factor of the movement in a pretreatment step performed before the medical procedure. An atlas of movements is constructed from the acquired images during the pretreatment step, which comprises the same sequence as the medical procedure but during which hyperthermia is not used. The acquisition of 50 images, for example, allows for a precise sampling of the respiratory cycle. The reference phase image that is selected is the first of the temporal series. During the pretreatment step, the anatomic images are stored in an atlas with the corresponding phase image. During the medical procedure, the current anatomic image is compared to the anatomic images stored in the atlas. The image in the atlas that is most similar is selected and the corresponding phase image is selected as a temperature calculation reference. The displacements of the organs are then estimated on the anatomic images in order to compensate both in the anatomic images as well as in the temperature images.

Furthermore, when the monitoring of the temperature variations and thus of the heat dosage to the target area, is done with precision, that is, by correcting for the temperature artifacts caused by the different movements of the target area, we may predict regulation means of the radiation applied to the treatment focal point P in the target area that is central to treatment device 1.

The heat images originating from the MRI imaging means 2 follow the heat dosage applied up until then to the target area, and it may be worthwhile to regulate the radiation of the target area at the treatment focal point P, so that the spatial distribution of temperature in the target area is in relation to a control signal for the spatial distribution of temperature.

The temperature measurement information originating from the temperature images allows for the precise control of the temperature of the target area, and to regulate it by using the regulation means so as to make it conform to the desired temperature in relation to the target area of the biological tissue.

When the target area can be treated in a spot procedure, the temperature may be adjusted from the MRI temperature images using a Proportional-Integral-Derived automatic regulation (PID). This technique is based on a differential equation [Equation 5] which includes a Proportional-Integral-Derived term, in order to minimize the error factor of temperature ξ.

$$\frac{\%}{t} + a \% + \frac{{a}^{2}}{4} {\#}_{0}^{t} \% = 0 with \% = ! T \qquad [ Equation 5 ]$$

The ξ parameter represents the difference between the target temperature θ and the measured temperature T.

The proportional term is equal to the instantaneous error between the measured temperature and the target temperature. The integral term is defined by the sum of the error and the past temperature. The derived term is determined by the variation present in the temperature so as to get the desired temperature.

The parameter may be defined by the operator and influence the relative importance of the three terms of the PID control. This automatic control ensures a stable convergence towards the target temperature.

The PID control is further combined with a temperature transfer equation [Equation 6] so as to predict the reaction of the biological tissue. This equation takes into account the temperature diffusion and absorption of the ultrasound wave by the tissue.

$$\frac{\# T}{\# t} = D ! {}^{2} T + ! P \qquad [ Equation 6 ]$$

The PID differential equation is respected if the power is chosen as per the value defined by the expression [Equation 7] so as to correctly adjust the temperature derived term:

$$P = \frac{1}{\% } \& \frac{{T}_{C}}{t} D ) ( {}^{2} T + a ( , T ) + \frac{{a}^{2}}{{4}^{t}} + {( , T )}_{!}^{\#} \qquad [ Equation 7 ]$$

To control the temperature on a determined point of the mobile tissue, the PID feedback control algorithm is calculated using the corrected temperature maps that are reset on a central reference position. With this temperature map, all of the calculations concerning the power required for temperature control are done in the same way as for static biological tissue. For each positioning of the treatment point, whether measured or predicted, the radiation power may then be adjusted.

To treat a large volume comprising several millimeters with a small focal point treatment of only one millimeter, one method consists in displacing the treatment focal point along a pathway. For a spatial control of temperature, the acoustic power quantity is defined by the equation [Equation 7] for all points of the target volume. However, whatever the technology that is used to displace the treatment focal point P (electronic or mechanical), it is technically difficult to produce this power and to simultaneously define it at each point. To avoid this problem, the quantity of energy that is transferred, E, during a feedback control cycle of time t_{F} at a point r is defined by the equation [Equation 8].

$${E}_{( \vec{r} )} = \frac{{t}_{F}}{ \%} \& , \frac{( \vec{r} )}{ t} D ) ( {}_{}^{2}{T}{}_{( \vec{r} )}^{} + a ( {,}_{( \vec{r} )} {T}_{( \vec{r} )} ) + \frac{{a}^{2}}{{4}^{t}} + {( {,}_{( \vec{r} )} {T}_{( \vec{r} )} )}_{!}^{\#} \qquad [ Equation 8 ]$$

This energy is delivered in succession to each point, with each duration and power value appropriately selected. The points requiring high levels of energy are heated over a long focus period rather than using high level of power so as to improve the security factor. As such the duration of the feedback cycle is divided into several lengths with a weighted value corresponding to the energy required to limit the maximum power emitted. In order to optimize the quality of the temperature control, the feedback cycle is selected so as to be as short as possible, that is, the duration of acquiring a dynamic.

The control means force the treatment focal point P to follow the movement of the target area, either by calculation or prediction, and thus the path that the treatment focal point P must follow for the treatment volume to be as large as possible, is also modified. Each point that defines this path is corrected in the same way which has been described here above, using the control means. In this way, the radiation is correctly regulated and sampled, and makes it possible to have an efficient volumetric treatment.

Assessment of the Treatment of Biological Tissues in Motion, with a Device According to the Invention

As illustrated in FIG. 3, the treatment device according to the invention was assessed for an ex-vivo pig muscle, made artificially mobile, places in an MRI imaging apparatus 11 and treated with focused ultrasounds using a transducer 12. The target area 13 is displaced at a distance by a transmission drive 14. The simulated movements are incidental if the drive is displaced manually or periodic if it is displaced by a motor 15. By making the voltage of the motor 15 vary it is possible to then select the periodicity of the movement. Furthermore, the movement of the target area 13 may be rigid if it is placed on a sliding or elastic guide if it placed in front of a stop.

In the case of the study of rigid movement, the translation of the target area 13, the position of the transmission drive is measured using a millimeter measuring strip 16. Two emitting photo diodes 17 and two photo receptors identify the position of the strip with a precision of 0.5 mm. Counting down the scale strip is done by a 20 MHz PIC microcontroller 18 which measures the target displacement time to the microsecond. After each change of the position of the scale strip, the time and position of the target are transmitted to a monitoring panel in a few microseconds using a RS232 connection for example. These time measurements of the position of the target 13 in real time serve as a reference to assess the quality of the estimated and predicted displacement using the images originating from the MRI.

### Correction of the Treatment Point

It is necessary at first to assess the performance of the treatment device with regard to the correction of movements of the target area. It is valuable to compare the estimated movement (which means, without the compensation for latency time) on the MRI images and the predicted movement (that is, both the predicted movement and the compensated movement taking into account the latency time) with the real movement measured by the microcontroller on the scale strip that is attached to the transmission drive.

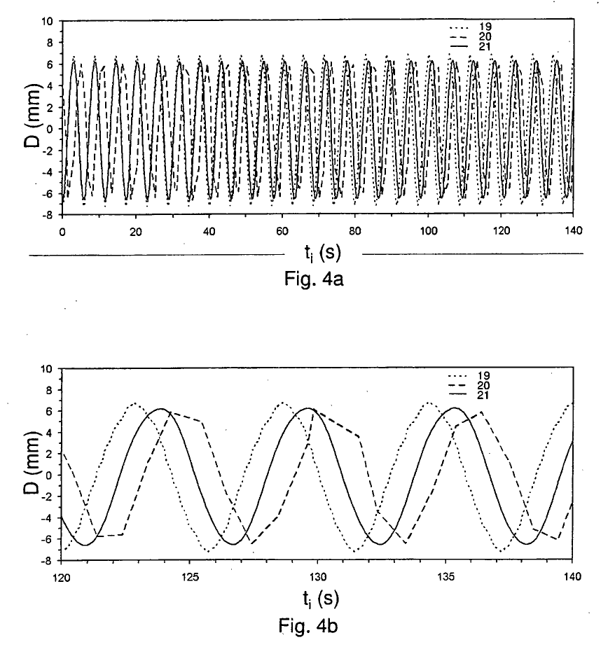

The first technique consists in supposing that the periodicity of the movement is perfectly constant throughout the procedure. A polynomial approximation of order 3 of the dominant predicted displacement of the pretreatment step is used to correct the movement. This polynomial coincides well with the real movement of the first dynamics. Nevertheless, despite the 1% variation in the angular speed of the lower motor, the predicted movement modeled for a polynomial function that is not re-updated, quickly diverges from real movement. FIGS. 4a and 4b show the real displacement (curve 19) measured on the scale strip, the estimated displacement (curve 20) of the anatomic images as well as the predicted displacement (curve 21) modeled for a constant polynomial function.

The standard deviation between the real movement and the predicted movement is very weak at the beginning of the experiment (0.33 mm) and increases rapidly up to 4.5 mm after 2 minutes. This technique shows that it is vital to re-update the prediction of the movement as frequently as possible.

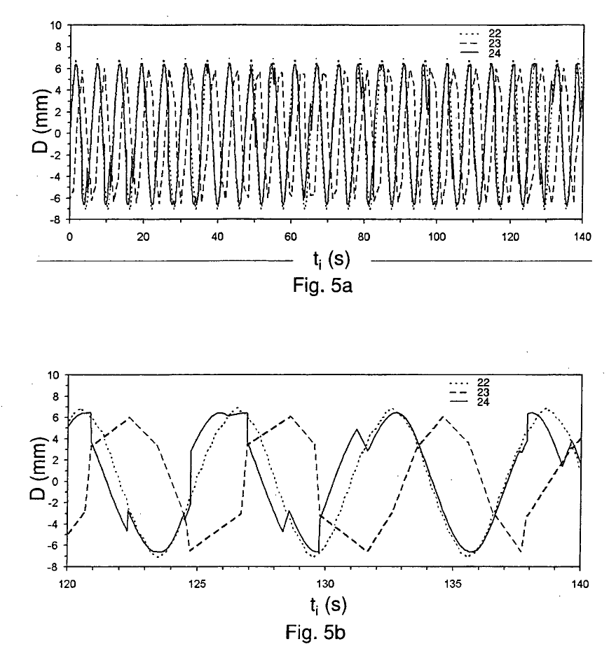

According to another assessment of the correction, the latency time is considered as constant. The ex-vivo muscle has been subjected in this experiment to a periodic translation movement of 14 mm of amplitude and of a period of about 5.6 s. On the testing platform used, the average latency time compensation is about 1.9 s. On FIGS. 5a and 5b this assessment is illustrated, with curve 22 representing the real displacement measured on the scale strip, and curve 23 showing the estimated displacement on the anatomic images and curve 24, showing the predicted displacement re-updated for each dynamic with a constant latency time value.

This method is more efficient than the preceding one because the standard deviation that is measured between the measured and the predicted movement oscillates between 1 mm at the beginning of the experiment and 2 mm at the end.

The time lag to be compensated is mainly composed of the acquisition duration for a dynamic (1 s) and the transmission and calculation times varying between 0.3 s and 1.1 s (average, 0.9 s). This variation of the information processing time is partly related to the use of an operation system which isn't a real time system. This variation of the transmission time induces a disturbance of the temporal location of the estimated movement which affects the predicted movement. As such, the predicted movement is composed of successive disjointed trigonometric curve.

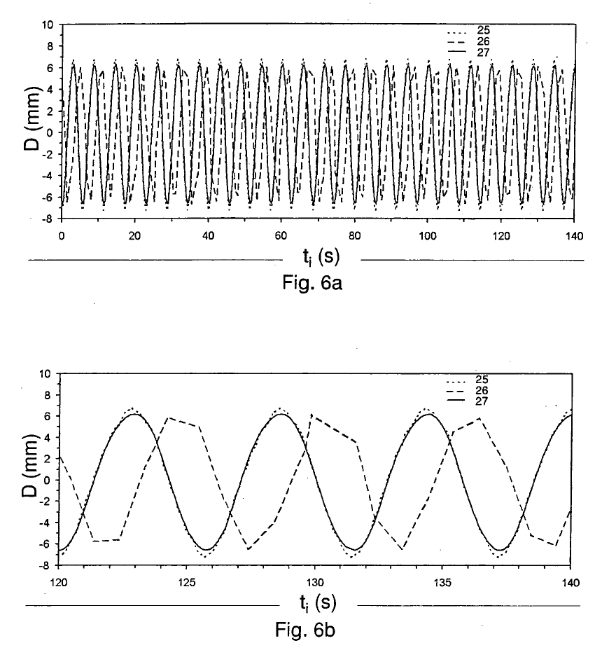

According to another assessment of the correction, the latency time to be compensated is obtained by comparing the time of the start of the last time-measured dynamic using a microcontroller, with the time of the end of the treatment. Similarly to the above, the ex-vivo muscle was subjected to a periodic translation movement of 14 mm amplitude and a period of about 5.6 s. On FIGS. 6a and 6b illustrating this assessment, curve 25 represents the real displacement measured on the scale strip, while curve 26 shows the estimated displacement on the anatomic images and curve 27 shows the predicted displacement re-updated for each dynamic with the measured latency time value.

This method is very efficient because the standard deviation measured between the measure and predicted movement is 0.33 mm. The predicted movement is continuous with the exception of minute discontinuities which adjust the periodicity of the movement.

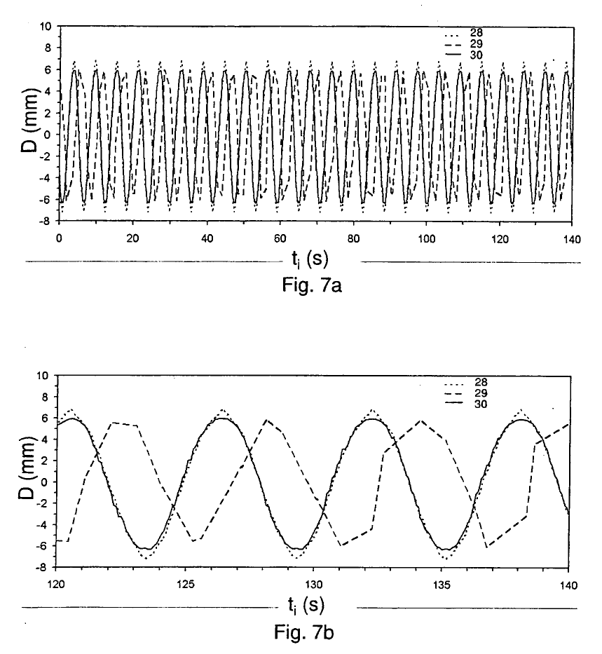

As indicated above, in order to correct the elastic movement, the dominant displacement is no longer enough. It shall nevertheless be used as a criteria for selecting appropriate vector fields from the atlas. FIGS. 7a and 7b show the predicted movement obtained using a technique referencing an atlas for the translation movement previously studied, with a latency time which is measured for each dynamic. Curve 28 represents the real displacement measured on the scale strip. Curve 29 shows the estimated displacement on the anatomic images and curve 30 shows the predicted displacement.

The predicted displacement is broken down into 50 values corresponding to the 50 vector fields stored in the atlas. This breakdown of the predicted movement only minutely diminished the precision of the determination of the movement. The standard deviation between the real movement and the predicted movement is 0.41 mm instead of 0.33 mm using the dominant movement.

With these different assessments one notices that when no correction is made, the average treatment focal point positioning error committed and measured after experimenting, is 4.76 mm.

The theoretical assessment of this error may be done by approximating the real movement with its first harmonic and by shifting the temporal point of origin. The equation thus becomes [Equation 1]:

$$M ( t ) {c}_{1} ! cos ( w t ) \\ with \\ {c}_{1} = \sqrt{{a}_{1} + {b}_{1}} = 6 , 5 mm \\ et \\ T = \frac{2}{\#} = 5 , 6 s \qquad [ Equation 9 ]$$

The theoretical error induced by this movement is therefore:

$$\begin{matrix}{\#}_{without correction} = \sqrt{\frac{1}{T} {}_{0}^{T}{( {c}_{1} ! cos ( w t ) )}{}_{}^{2} d t} \\ = \frac{{c}_{1}}{\sqrt{2}} \\ = 4 , 6 mm\end{matrix} \qquad [ Equation 10 ]$$

When the focal point is positioned according to the estimated movement of the last available image, the experimental error reading is on average 7.54 mm.

The theoretical estimate of this error may be made by taking the latency time compensation as a constant (1.9 s). The induced phase difference is:

$$c = 2 ! \frac{{d}_{C}}{{T}^{′}} = 2 , 1 rad = 120 ° \qquad [ Equation 11 ]$$

The theoretical error induced by this phase difference is:

$$\begin{matrix}{\%}_{without prediction} = \sqrt{\frac{1}{T} {\#}_{0}^{T} {( {c}_{1} ! cos ( w t ) {c}_{1} ! cos ( wt {+}_{C} ) )}^{2} d t} \\ = {c}_{1} ! \sqrt{2} ! \vert sin \frac{ }{2} \vert \\ = = 8 mm\end{matrix} \qquad [ Equation 12 ]$$

When the compensated latency time is correctly measured, the measured experimental error is 0.33 mm. In theory, there is no more phase difference between the predicted movement and the real movement:

!_{predicted}=0 [Equation 13]

The following table compares the theoretical and experimental standard deviations obtained:

| | | | |
|---|---|---|---|
| Standard | | Correction | Correction |
| deviation | No correction | No prediction | Prediction |

| | | | | | | |
|---|---|---|---|---|---|---|
| Experimental | 4.76 | mm | 7.54 | mm | 0.33 | mm |
| Theoretical | 4.6 | mm | 8 | mm | 0 | mm |

The predicted movement allows for a positioning precision of the treatment focal point at least 14 times more precise than a positioning without movement correction.

Finally, one may note that the correction of periodic movements is only for the correction of movements with a period which is greater than the acquisition duration of a dynamic. Indeed, as per the Shanon-Nyquist theorem, a sampling of at two least dynamics per period is necessary in order to be able to reconstruct a complete period. In the experiments presented, the period of the movement that was made was about 5.6 seconds, therefore the acquisition of a one dynamic per second made possible a good reconstitution of the movement.

### Correction of Temperature Measurement Artifacts in Addition to the Correction of the Treatment Point

It is therefore necessary to then assess the performances of the treatment device when it is able to correct the temperature measurement artifacts due to movements of the target area. We therefore heated the ex-vivo muscle using a focused ultrasound device while subjecting it successively to different types of movement during the heating process.

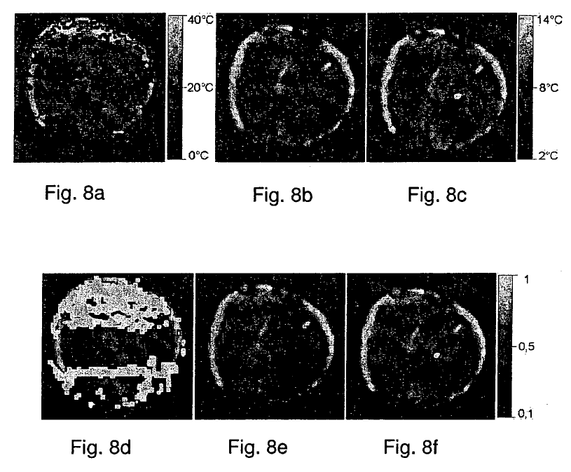

The ex-vivo muscle at first was subjected to an incidental translation movement of 14 mm. The muscle was heated by the ultrasound transducer emitting a power of 75 W of electricity for 50 seconds. The incidental movement was performed at the half-way point of the heat dosage (at the end of the experiment). FIGS. 8a to 8c represent the temperature of the maps without temperature measurement or treatment point position corrections, with temperature measurement corrections but without treatment point position corrections, and finally, with temperature measurement and treatment point position corrections. Likewise, FIGS. 8d to 8f represent the maps of the heat measurement respectively without temperature measurement and treatment point position corrections, with temperature measurement corrections but without treatment point position corrections and finally, with temperature measurement and treatment point position corrections.

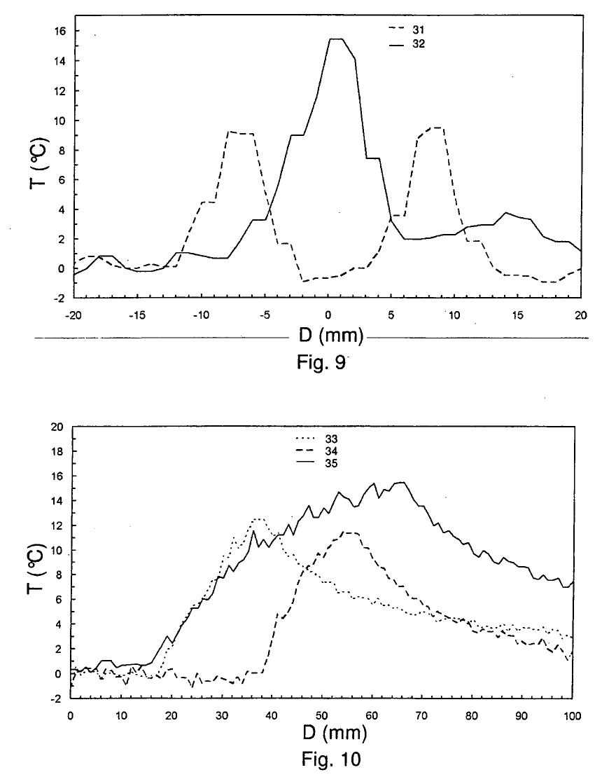

FIG. 9 shows the spatial distribution of the temperature along the vertical axis passing through the focal points presented on the temperature maps, with (curve 32) and without (curve 31) treatment point position corrections.

FIG. 10 shows the temporal changes of temperature in the heated zones during the experience with (curve 35) and without (curve 33 prior to movement, and curve 34 after movement) treatment point position correction.

The temperature error committed without correction of the temperature measurement, may reach 40° C. This artifact completely masks the heating process. Likewise, the heat dosage that is calculated cannot be used.

If no correction of the treatment focal point is made, two heated zones appear simultaneously: the previously heated zone which is cooling, and the new target heating zone. These two zones are spaced 14 mm apart as per the movement that was made. Neither of them leads to tissue necrosis because the local accumulation of energy wasn't sufficient enough.

When the displacement of the treatment focal point compensates the target motion, a heated zone spot is observed. The continuous logarithmic form of the rising temperature shows that the heating isn't affected by the movement. Likewise, the circular form shows that the movement was properly corrected. The energy was still latent on the same tissue area, and a necrosis was induced.

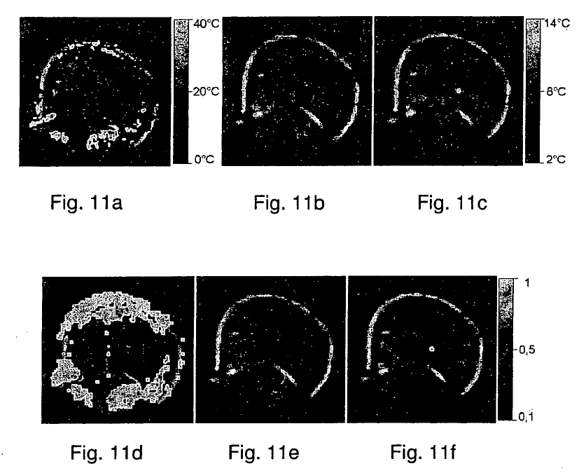

The ex-vivo muscle was then subjected to a periodic translation movement of 14 mm amplitude with a period equal to about 5.6 seconds. The muscle was heated by the ultrasound transducer emitting a power of 100 W of electricity for 1 minute. FIGS. 11a to 11f compare the temperature maps (FIGS. 11a to 11c) and heat dosage (FIGS. 11d to 11f), without temperature measurement and treatment focal point position correction (FIGS. 11a and 11d), with temperature measurement but without treatment focal point position correction (FIGS. 11b and 11e), and finally with correction of the temperature measurement and the treatment focal point position (FIGS. 11c and 11f).

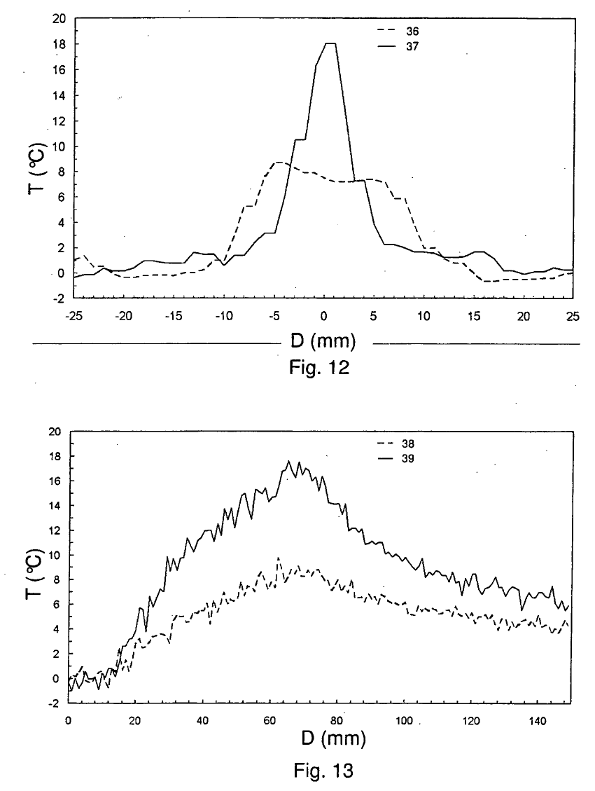

FIG. 12 shows the spatial distribution of the temperature along the vertical axis (corresponding to the axis of the muscle movement) passing through the heated zones presented on the temperature maps, with (curve 37) and without (curve 36) correction of the treatment point position.

FIG. 13 shows the temporal change in the temperature of the heated zones during this experiment with (curve 39) and without (curve 38) correction of the treatment point position.

As above, the temperature error committed without correction of the temperature measurement can reach 40° C. This artifact completely masks the heating that has been done. Likewise, the same heat dosage that is calculated cannot be used.

When no correction of the focal point is done, the heated zone is spread over 14 mm. The induced heating doesn't lead to tissue necrosis because the rise in temperature is insufficient.

When the focal point displacement compensates the target movement, a circular heating zone shows that the movement was properly corrected. The continuous logarithmic form of the rising temperature shows that the heating isn't affected by the movement. The temperature reached by the tissue was two times as high as above. The energy was still latent on the same tissue area, and a necrosis was induced.

The most frequent case and the most complicated to correct is the case of elastic periodic movement. In order to study such displacements, the vivo muscle was subjected to a crushing periodic movement with a periodicity of about 5.6 s. The muscle was heated by the ultrasound transducer emitting a power of 100 W of electricity for 1 minute.

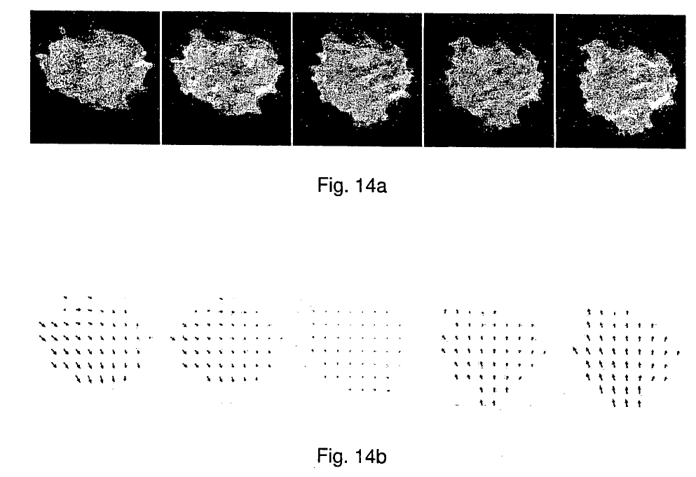

FIGS. 14a and 14b respectively show the anatomic images obtained at different movement positions with the associated vector field displacements, estimated as per a central reference image.

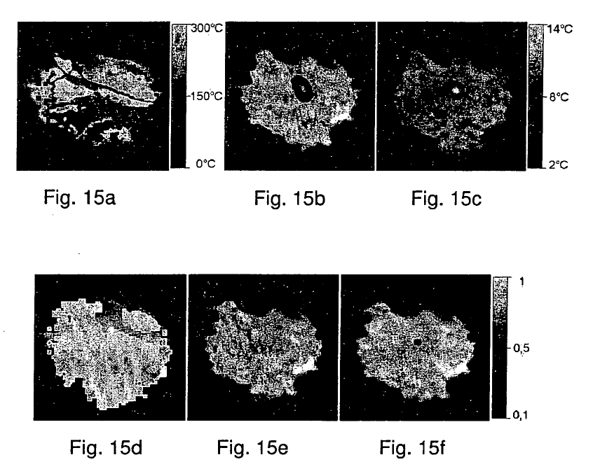

FIGS. 15a to 15f compare the temperature maps (FIGS. 15a to 15c) and heat dosage (FIGS. 15d to 15f), without temperature measurement and treatment focal point position correction (FIGS. 15a and 15d), with temperature measurement correction but without treatment focal point position correction (FIGS. 15b and 15e), and finally with temperature measurement and treatment focal point position correction (FIGS. 15c and 15f).

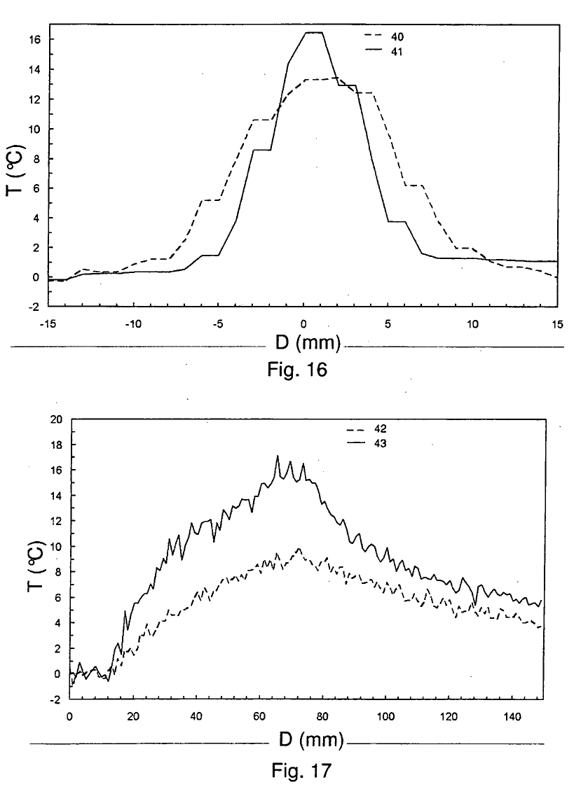

FIG. 16 shows the spatial distribution of temperature along a vertical axis passing through the heating zones presented on the temperature maps, with (curve 41) and without (curve 40) correction treatment point position.

FIG. 17 shows the temporal change of the temperature in the heated zones throughout the experiment with (curve 42) and without (curve 43) correction of the treatment point position.

The temperature error committed without correction of the temperature measurement can reach 300° C. in 1 minute. Phase changes that are greater than 2π appear between two successive dynamics, which induces a temperature error which accumulates over time. This artifact completely masks the heating. Likewise, the heat dosage that is calculated cannot be used.

When there is no correction of the focus point, the heating zone is spread over 12 mm. The heating that is induced doesn't lead to necrosis of the tissues because the rise in temperature is not sufficient to do so.

When the displacement of the focusing point compensates the target movement, a circular heating zone shows that the movement was corrected accurately.

The continuous logarithmic form of the rising temperature shows that the heating isn't affected by the movement. The temperature reached by the tissue is higher than it was previously. The energy was still latent on the same tissue area, and a necrosis was induced.

Control Feedback of the Temperature with Correction of the Treatment Point Position

The heat treatment device with monitoring of the displacement presented here above allows for focusing, using constant power that is always focused on the desired point regardless of the position of the tissue. Furthermore, the temperature measurement correction renders quality temperature maps that are as good as those obtained without movement. These corrected temperature maps may help to perform a control feedback of the temperature as presented here above. In this way, the spot and spatial temperature control techniques were applied to ex-vivo muscle subjected to periodic rigid movement.

In order to control temperature on affixed point of mobile tissue, the spot temperature feedback control algorithm PID described above is performed using the corrected temperature maps reset on a central position of movement. As such, all of the calculations concerning the power necessary to provide a temperature feedback control are made as when the tissue is immobile. Once the required power is determined, the position of the focusing point is adjusted every 100 ms using the predicted algorithm of the periodic movement described above.

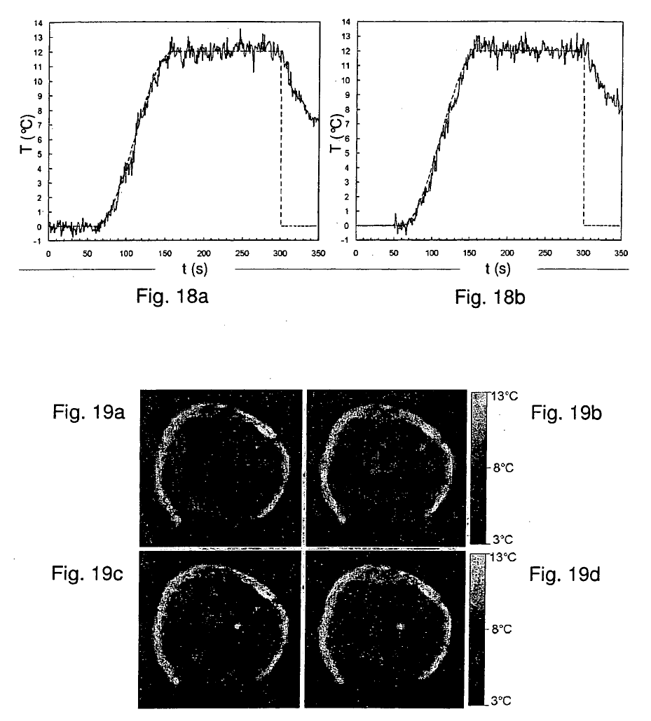

FIGS. 18a and 18b compare the control of temperature on immobile ex-vivo muscle (FIG. 18a) and subjected to a periodic rigid movement (FIG. 18b). The movement of period 6 s and amplitude 14 mm is reconstructed on a pretreatment step of 50 dynamics. As above, each dynamic is acquire din 1 s with voxels of 1.5×1.5×4.5 mm³.

The response time that is selected to perform the control feedback is 8 s. The tissue parameters that are used to predict the tissue behavior using a Fourrier transformation are 0.1 mm²/s for the diffusion coefficient and 0.006K/J for the absorption coefficient.

The set temperature of 12° C. between 160 s and 300 s is reached both with and without tissue movement within a range of 0.45° C. Given the fact that the intrinsic noise of the sequence used is 0.3° C., the correction of the temperature measurement and the feedback control calculations of the temperature induce very little noise on the rising temperature that is obtained.

FIGS. 19a to 19d represent temperature maps corresponding to 114 s dynamics (FIGS. 19a and 19b) and 183 s dynamics (FIGS. 19c and 19d) of the two spot control feedbacks performed without movement (FIGS. 19a and 19c) and with movement (FIGS. 19b and 19d).

Thanks to the techniques for image resetting and temperature measurement artifact correction, the acquired dynamics of the tissue in motion are difficult to differentiate from those acquired on the immobile tissue. Furthermore, both cases have the same temperature isovalues, as indicated by the circular forms around the focusing point. This shows that the focusing point is correctly positioned on the inside of the tissue regardless of the displacement. In the contrary case, the heating extends in the direction of the movement (as in FIG. 11b).

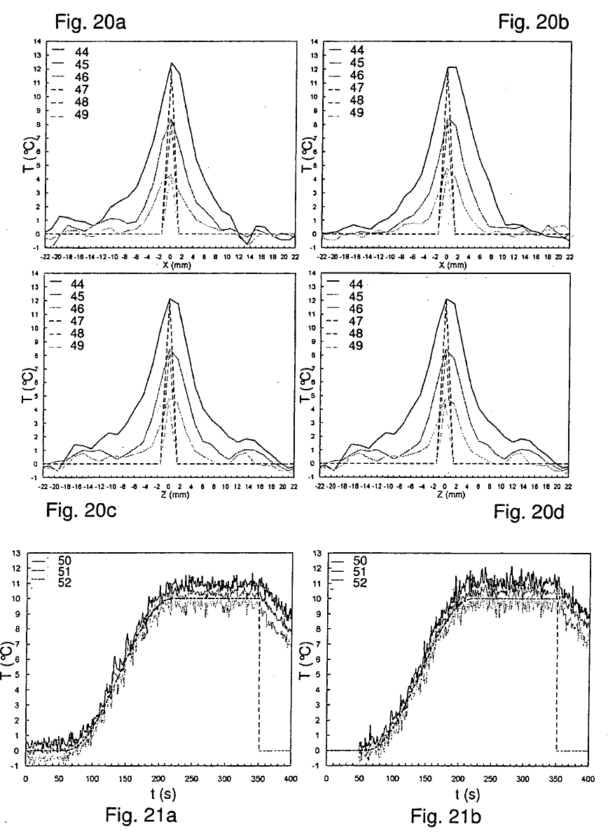

To quantify with better precision the spatial distribution of the temperature between these two experiments, FIGS. 20a to 20d represent the temperature along the two axes X and Z on an immobile ex-vivo muscle (FIGS. 20a in X and 20c in Z) or else subject to a rigid periodic movement (FIGS. 20b in X and 20d in Z), for the dynamics 103 s (curve 46), 124 s (curve 45) and 178 s (curve 44).

It is possible to note that on each of these graphs that the set temperatures (curve 49 at 103 s, curve 48 at 124 s and curve 47 at 178 s) are respected as per the precision. Furthermore, the treatment focal point was correctly reset since the width of the heating is the same along axes X and Z even if a 14 mm movement is noticed along axis Z.

On the other hand, by comparing the heating without movement with the heating subjected to movement, the spatial distribution of the temperature increase about 10% for axes X and Z. This broadening appears simultaneously on both axes and cannot be explained by a lack of precision in the positioning of the treatment focal point but rather by the addition of secondary lobes related to the electronic displacement of the treatment point. Other than these secondary lobes, the temperature control with movement monitoring works as well on a mobile as on an immobile tissue.

Likewise as for the case of spot feedback control of temperature, we can perform a spatial feedback control of temperature on mobile tissue. As described above, we use a corrected and reset temperature map. The path provided by the spatial feedback control algorithm of temperature is then modified so as to make the focusing points coincide with those selected from the reset image. For this, each pathway point is subdivided into a series of points whose duration is close to 100 ms. Each one of these points is then translated as per the predicted value of the calculated movement, like with the other heating processes. In this way, the energy that is deposed on each of the points defined by the temperature control algorithm and the position of the focusing points is adjusted according to the movement with good temporal sampling.

FIGS. 21a and 21b compare the two heating processes performed with a spatial feedback control of temperature on a both immobile tissue or else in motion with periodic translation. The movement amplitude is reduced to 8 mm along the Z axis so that the focusing point may be deviated to a range of 9 mm in the perpendicular direction, along axis X. In this way, even when the tissue is located in an extreme position of the movement, the focusing point doesn't deviate more than 6 mm from its central position. The width of the voxels being 1.5 mm, the temperature control was performed on the 7 central voxels of the Z axis, that is, a segment width of 9 mm. Curves 52, 51, and 50, respectively represent the minimum, average and maximum value of the temperature on these 7 voxels in relation to the set temperature.

The rise in temperature follows the set level through the whole control zone with a precision of 0.5° C. The difference between the maximum temperature and the minimum temperature on the 7 feedback control voxels is 1.2° C. for the immobile tissue and 1.3° C. for the mobile tissue. With the movement monitoring technique, the displacement of the tissue hardly introduces any noise in the temperature measurement or on the precision of the heating control.

FIGS. 22a to 22d represent the temperature maps corresponding to dynamics of 141 s (FIGS. 22a and 22b) and 215 s dynamics (FIGS. 22c and 22d) of these two spot temperature feedback controls performed without movement (FIGS. 22a and 22c) and with movement (FIGS. 22b and 22d). The elongated segment of 9 mm along the X axis appears as very distinct even if the tissue is subjected to a periodic movement of 8 mm in a perpendicular direction. Since the heated segment isn't deformed, the focusing points can follow the movement well.

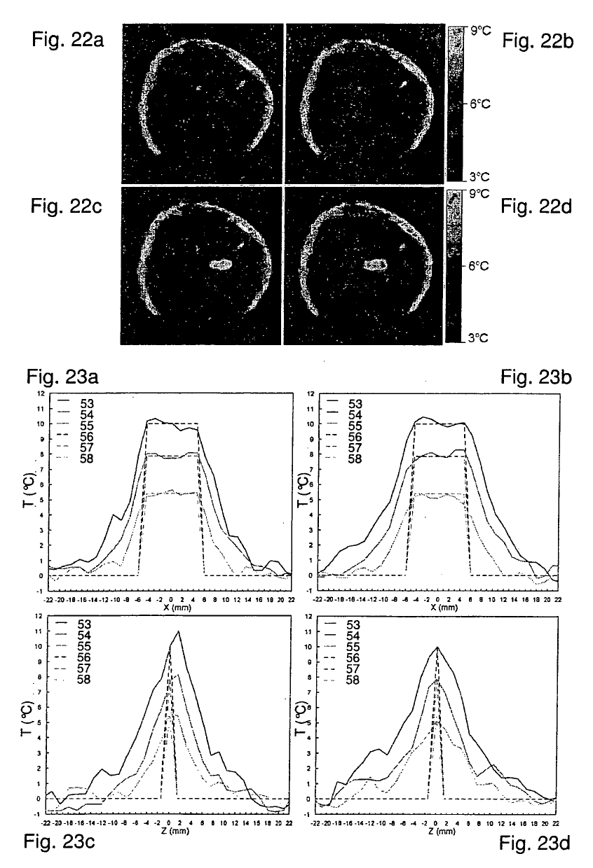

For a more detailed observation of the spatial control of temperature, FIGS. 23a to 23d represent the temperature along two axes X and Z on an immobile ex-vivo muscle (FIGS. 23a on X and 23c on Z) or else subject to a rigid periodic movement (FIGS. 23b on X and 23d on Z), for dynamics of 145 s (curve 55), 172 s (curve 54) and 219 s (curve 53).

The rise in temperature thus obtained corresponds to a set temperature plateau (curve 58 at 145 s, curve 57 at 172 s and curve 56 at 219 s) along axis X for each dynamic even if the heat diffusion effect counteracts it, or if the tissue is displaced along a direction perpendicular to axis Z. As above, the spatial distribution of heating on the tissue in motion is slightly greater along axes X and Z in comparison to the heating on immobile tissue. This can also be explained by the presence of lobes that are larger as the focusing point draws further from its central position.

Furthermore, for heating performed on immobile tissue the focusing point is shifted one half-voxel along the Z axis. This slight shift with no serious consequences occurs frequently since the spatial resolution of the MRI images that are used is expressly dimensioned as close as possible to the treatment point dimensions.

The reader may notice that numerous modifications may be made without departing from the material facts related here regarding to the matter that has been explained and the advantages thereof. As a consequence, all of the modifications of this sort are meant to be incorporated into the scope of the heat treatment device for biological tissue in movement according to the invention, and to the related treatment method associated the heat treatment method.

1. A heat treatment device of a target area in motion of biological tissue, comprising calculation means (6) for estimating a position of the target area using a measurement signal of the target area, wherein it further comprises controlling means (7) for positioning a treatment focal point (P) in the target area based on the estimated position and a positioning time lag between a measurement of the measurement signal of the target area and the positioning of the treatment focal point (P), so as to compensate the movement of the target area during the positioning time lag.

2. The device of claim 1, wherein the positioning time lag comprises a latency time due to the estimation of the position of the target area using the calculation means (6), so that the control means (7) are adapted for compensating the movement of the target area during the latency time.

3. The device of any of claim 1, wherein the positioning time lag comprises a prediction time of the movement of the target area, so that the control means (7) are adapted for predicting and compensating the movement of the target area during the prediction time.

4. The device of claim 2 further comprising measurement means (10) of the latency time to transmit to the control means (7).

5. The device of claim 1, further comprising modeling means for modeling the movement of the target area based on a series of measurement signals of the target area.

6. The device of claim 5, wherein the modeling of the movement is periodic.

7. The device of claim 5, wherein the modeling means comprise means to provide a spatial position of the target area based on a temporal position, with the spatial and temporal positions defining the position of the target area.

8. The device of claim 7, wherein the calculation means (6) comprise means to determine an estimated spatial position of the target area using a algorithm for processing of the measurement signal of the target area.

9. The device of claim 8, wherein the calculation means (6) comprise means to determine, according to the modeling of the movement of the target area, an estimated temporal position of the target area corresponding to the estimated spatial position.

10. The device of claim 9, wherein the control means (7) comprise means to position the treatment focal point (P) in accordance with a real temporal position of the target area, the real spatial position being based on the real temporal position according to the modeling of the movement of the target area, the real temporal position corresponding to the estimated temporal position enhanced by the latency time.

11. The device of claim 10, wherein the control means (7) further comprise means to position the treatment focal point (P) between successive estimations performed by the calculation means (6) of a first and second temporal position estimated respectively from a first and second measurement signal of the target area, in order to predict the movement of the target area.

12. The device of claim 11, wherein the control means (7) comprise means to position the treatment focal point (P) according to a predicted spatial position, the predicted spatial position being a factor of a predicted temporal position according to the modeling of the movement of the target area, the predicted temporal position corresponding to the estimated temporal position enhanced by the latency time and the prediction time.

13. The device of claim 1, wherein the calculation means (6) comprise means to determine an estimated displacement vector field of the target area using an algorithm for processing of the measurement signal of the target area.

14. The device of claim 13, wherein the control means (7) comprise means to position the treatment focal point (P) in the target area as a factor of the estimated displacement vector field.

15. The device of claim 1, further comprising imaging means (2) comprising means to measure the measurement signal of the target area and means to provide an anatomic image of the target area using the measurement signal of the target area.

16. The device of claim 15, wherein the imaging means (2) further comprise means to provide a phase image of the target area using the measurement signal of the target area, for monitoring temperature variations of the target area using a reference phase image.

17. The device of claim 16, wherein the calculation means (6) further comprise means to modify the reference phase image to correct a disturbance of the temperature due to a movement of the target area.

18. The device of claim 17, wherein the calculation means (6) comprise means to modify the reference phase image using stored phase images.

19. The device of claim 15, further comprising regulation means of a radiation applied on the treatment focal point (P) in the target area so that the spatial distribution of temperature of the target area should conform with a setting for the spatial distribution of temperature.

20. The device of claim 19, wherein the regulation means comprise means to regulate the applied radiation as a factor of the spatial distribution of temperature in the target area and a setting for the spatial distribution of temperature, in accordance with a regulation equation comprising a Proportional-Integral-Derived term.

21. The device of any of the preceding claims, wherein the heat treatment of the target area is non-invasive.

22. A heat treatment method of a target area in motion of a biological tissue, comprising the steps of:
Measuring the target area for obtaining a measurement signal of the target area,
Estimating a position of the target area based on the measurement signal of the target area using calculation means (6),
Positioning a treatment focal point (P) in the target area using control means (7), in based on the estimated position and the positioning time lag between the measurement of the measurement signal of the target area and the positioning of the treatment focal point, so as to compensate the movement of the target area during the positioning time lag.

23. The method of claim 22, wherein the positioning time lag comprises a latency time due to the estimation of the position of the target area using the calculation means (6), so as to compensate the movement of the target area during the latency time.

24. The method of any of claim 22, wherein the positioning time lag comprises a prediction time of the movement of the target area, so as to predict and compensate the movement of the target area during the prediction time.

25. The method of any claim 23, further comprising the step of measuring the latency time in real time using measurement means (10).

26. The method of claim 22, further comprising the step of modeling the movement of the target area based on a series of measurement signals of the target area.

27. The method of claim 26, wherein a period for modeling the movement of the target area is determined.

28. The method of claim 26, wherein the position of the target area is modeled using a spatial position as a factor of a temporal position.

29. The method of claim 28, wherein the estimation of the position of the target area comprises the steps of:
Determining the estimated spatial position of the target area using an algorithm for processing the measurement signal of the target area, and
Determining, according to the modeling of the movement of the target area, an estimated temporal position corresponding to the estimated spatial position.

30. The method of claim 29, wherein the positioning of the treatment focal point (P) comprises the steps of:
Determining a real temporal position of the target area, the real temporal position corresponding to the estimated temporal position enhanced by the latency time,
Determining a real temporal position of the target area, according to the modeling of the movement of the target area, based on the real temporal position, and
Positioning the treatment focal point (P) according to the real spatial position.

31. The method of claim 30, wherein the positioning of the treatment focal point (P) comprises the additional steps of:
Determining a predicted temporal position of the target area, the predicted temporal position corresponding to the estimated temporal position enhanced by the latency time and a prediction time,
Determining a predicted spatial position of the target area, according to the modeling of the movement of the target area, based on the predicted temporal position, and
Positioning the treatment focal point (P) based on the predicted spatial position.

32. The method of claim 31, wherein the additional steps for positioning the treatment focal point (P) are repeated until a new estimated temporal position is determined.

33. The method of any of claim 24, further comprising the step of determining an estimated displacement vector field of the target area using an algorithm for processing the measurement signal of the target area.

34. The method of claim 33, wherein the treatment focal point is positioned in the target area based on the estimated displacement vector field.

35. The method of claim 24, wherein the measurement signal of the target area provides an anatomic image of the target area for positioning the treatment focal point.

36. The method of claim 35, wherein the measurement signal of the target area further provides a phase image of the target area.

37. The method of claim 36, further comprising the steps of:
Determining a reference phase image;
Comparing the acquired phase image and the reference phase image for monitoring the temperature variations in the target area.

38. The method of claim 37, wherein a reference phase image is determined from the stored phase images.

39. The method of claim 38, further comprising the step of regulating the radiation applied to the treatment focal point (P) in the target area so that the spatial distribution of temperature in the target area conforms with a setting for the spatial distribution of temperature.

40. The method of claim 39, wherein the radiation applied is regulated based on the spatial distribution of the temperature in the target area and the setting for the spatial distribution of temperature, according to a regulation equation comprising a Proportional-Integral-Derived term.

41. The method of any of claims 22 to 40, wherein it is performed non-invasively.
